(19) United States
(12) Patent Application Publication
(10) Pub. No.: US 2017/0349882 A1
(43) Pub. Date: Dec. 7, 2017

(54) CELL CULTURE SUBSTRATE FOR TRAIT INDUCTION CONTROL OF MACROPHAGE AND METHOD OF CONTROLLING TRAIT OF MACROPHAGE

(71) Applicant: Tokyo Ohka Kogyo Co., Ltd., Kanagawa (JP)

(72) Inventors: Emi MAENO, Kawasaki-shi (JP); Takahiro SENZAKI, Kawasaki-shi (JP); Atsushi MUROTA, Kawasaki-shi (JP); Yasuhiko TABATA, Kyoto (JP)

(73) Assignee: Tokyo Ohka Kogyo Co., Ltd., Kanagawa (JP)

(21) Appl. No.: 15/610,733

(22) Filed: Jun. 1, 2017

(30) Foreign Application Priority Data
Jun. 2, 2016 (JP) 2016-110924

(51) Int. Cl. C12N 5/0786

(52) U.S. Cl. CPC C12N 5/0645; C12N 2535/10

(57) ABSTRACT

Provided is a cell culture substrate for trait induction control of a macrophage, which has a pattern of unevenness on a surface to which a cell adheres, the width of the unevenness being 50 nm or more and less than 1,000 nm.

### BACKGROUND OF THE INVENTION

### Field of the Invention

The present invention relates to a cell culture substrate for trait induction control of a macrophage and a method of controlling trait of a macrophage.

Priority is claimed on Japanese Patent Application No. 2016-110924, filed on Jun. 2, 2016, the content of which is incorporated herein by reference.

### Background Art

A macrophage (expressed as MΦ by the symbol) is known to differentiate from a monocyte (mononuclear leukocyte) occupying 5% of leukocytes in the blood. The monocyte within tissues differentiates into the macrophage under influences of various environmental factors and inducers in each tissue. It is known that there are at least two kinds of M1 (inflammatory type) macrophage trait-induced by stimulation of Th1 cytokines such as interferon (IFN)-γ, tumor necrosis factor (TNF)-α, lipopolysaccharide (LPS), and the like, and M2 (anti-inflammatory type) macrophage trait-induced by Th2 cytokines such as interleukin (IL)-4, IL-13, and the like in the differentiated types of macrophages. It is known that a resting macrophage, as well as the active macrophages (M1 and M2), is present as the macrophages.

It is considered that the M1 macrophage has high expression levels of inflammatory cytokines such as TNF-α and IL-1, and the like, induces oxidative stress and induces neutrophil infiltration via secretion thereof, decompose necrotic tissues, removes foreign substances and bacteria, and plays a part thereof by its own phagocytosis. On the other hand, it is considered that the M2 macrophage highly expresses IL-10, transforming growth factor (TGF)-β and the like, acts in a direction to suppress inflammation through reduction of inflammatory cytokine secreted from the M1 macrophage via IL-10 secretion, and is involved in tissue repair via secretion of TGF-β, platelet-derived growth factor (PDGF), vascular endothelial cell growth factor (VEGF), and the like.

In this manner, the M1 macrophage and the M2 macrophage play different roles in vivo, and in order to function properly the mechanism from inflammation induced by external stimulation to damage repair thereof, the balance between the M1 macrophage and the M2 macrophage is important. Therefore, an imbalance in the balance between the M1 macrophage and the M2 macrophage is considered to a cause of various diseases or disorders.

Japanese Unexamined Patent Application, Publication No. 2014-181191 discloses a trait inducer to the M2 macrophage containing lactic acid bacteria.

### SUMMARY OF THE INVENTION

In the method of using lactic acid bacteria as a trait inducer, the lactic acid bacteria used must be removed after a macrophage differentiates into a M2 macrophage. In addition, it is possible to induce trait into the M2 macrophage, but it is not possible to induce trait into a M1 macrophage.

The present invention has been made in view of the above circumstances, and an object thereof is to provide a cell culture substrate that easily and efficiently induces a macrophage to a certain trait.

The present inventors paid attention to the fact that the trait induction of the macrophage is due to the stimulation by nanoscale molecules on the cell surface, and the present invention has been completed.

That is, the present invention includes the following aspects.

According to a first aspect of the present invention, there is provided a cell culture substrate for trait induction control of a macrophage, which has a pattern of unevenness on a surface to which a cell adheres, the width of the unevenness being 50 nm or more and less than 1,000 nm.

According to a second aspect of the present invention, there is provided a method of controlling trait of a macrophage, including culturing a macrophage on the cell culture substrate for trait induction control of a macrophage.

According to the cell culture substrate for trait induction control of the present invention, the macrophage can be easily and efficiently induced to a certain trait. In addition, it can be applied to medical materials with improved biocompatibility. According to the method of controlling a trait of a macrophage of the present invention, the macrophage can be induced into an inflammatory type or an anti-inflammatory type macrophage, and disease models and the like can be easily constructed.

### BRIEF DESCRIPTION OF THE DRAWINGS

FIG. 1A is an enlarged plan view and an enlarged front view of a cell culture substrate (pattern: line shape) for trait induction control of a macrophage according to an embodiment. FIG. 1B is an enlarged plan view and an enlarged front view of a cell culture substrate (pattern: dot shape) for trait induction control of a macrophage according to the embodiment.

FIGS. 2A to 2D are graphs illustrating quantitative results of the amount of nitric oxide (NO) production of RAW264 cells in Example 1.

FIGS. 3A to 3D are graphs illustrating measurement results of arginase activity of RAW264 cells in Example 1.

FIGS. 4A and 4B are images illustrating results of a trait induction test on foam cells of RAW264 cells in Example 2.

### DETAILED DESCRIPTION OF THE INVENTION

Cell Culture Substrate for Trait Induction Control of Macrophage

In an embodiment, the present invention provides a cell culture substrate for trait induction control of a macrophage includes a pattern of unevenness on a surface to which a cell adheres, the width of the unevenness being 50 nm or more and less than 1,000 nm.

According to the cell culture substrate for trait induction control of a macrophage of the embodiment, the macrophage can be easily and efficiently induced to a certain trait. In addition, it can be applied to medical materials with improved biocompatibility. In addition, by using the cell culture member for trait induction control of a macrophage of the present embodiment, the macrophage can be induced into an inflammatory type (M1) macrophage, an anti-inflammatory type (M2) macrophage, a foam cell, or the like. In other words, the method of the present embodiment is a method of controlling the proliferation, differentiation and transformation of a macrophage. Therefore, in the present specification, “trait induction” includes “induction of differentiation”.

Monocyte

In the present specification, “monocyte” is an immature phagocyte circulating in the blood, and refers to a cell serving as an antigen-presenting immune cell, having a function of capturing and decomposing a pathogen and a foreign substance by phagocytosis.

As the monocyte, for example, the monocyte derived from mammals such as mouse, monkey, and human may be used. Among these, a human-derived monocyte is preferable, and a human peripheral blood-derived monocyte (human peripheral blood monocyte) is more preferable.

For the monocyte, a commercial product such as CD14⁺ monocyte (manufactured by Promocell Corporation) derived from human peripheral blood may be used, or a monocyte collected from apheresis of peripheral blood (whole blood) collected from a donor may be used. The method of apheresis is not particularly limited as long as the method can separate the monocyte from whole blood, and for example, a blood component separation device or the like may be used. Examples of the blood component separation device include COBE® Spectra (manufactured by Terumo BCT, INC), COM.TEC (manufactured by Fresenius Kavi Co., Ltd.), and the like. In addition, the monocyte may be separated and collected from whole blood by density gradient centrifugation without using the blood component separation device. The donor is a mammal (preferably human), and in a case of collecting blood from a donor, granulocyte colony stimulating factor (G-CSF) may be administered to the donor several days before blood collection.

Macrophage

In the present specification, “macrophage” refers to a cell positive for CD14 serving as a marker of the macrophage. Furthermore, the macrophages can be classified into an active macrophage including the M1 (inflammatory type) macrophage or the M2 (anti-inflammatory type) macrophage, and a resting macrophage according to the differentiated type thereof.

The macrophage used for culturing using the cell culture substrate for trait induction control of a macrophage of the embodiment is preferably the resting macrophage, and may be the resting macrophage induced to differentiate from the above-described monocyte. In addition, a trait-induced macrophage can be obtained by using the cell culture substrate for trait induction control of a macrophage of the embodiment. The macrophage obtained is preferably the active macrophage, and more preferably the M1 macrophage or the M2 macrophage.

Hereinafter, the active macrophage, the M1 macrophage, the M2 macrophage, and the resting macrophage will be described.

Active Macrophage

The active macrophage refers to a cell positive for CD14, and positive for CD80 serving as a marker of the M1 macrophage or CD206 serving as a marker for the M2 macrophage. That is, the active macrophages include the M1 macrophage and the M2 macrophage.

M1 (Inflammatory Type) Macrophage

The M1 macrophage refer to a cell positive for CD14 serving as the marker of the macrophage and positive for CD80 serving as the marker for the M1 macrophage. The M1 macrophages are known as a classical activated macrophage and an inflammatory macrophage, and are considered to enhance immunity and to induce and promote inflammation.

M2 (Anti-Inflammatory Type) Macrophage

The M2 macrophage refers to a cell positive for CD14 serving as the marker of the macrophage and positive for CD206 serving as the marker for the M2 macrophage. The M2 macrophages are known as a wound healing macrophage and an anti-inflammatory macrophage, and are considered to suppress immunity and to direct inflammation towards termination.

Resting Macrophage

The resting macrophage refers to a cell positive for CD14 serving as the marker of the macrophage and negative for both CD80 and CD206. The resting macrophage is considered that the monocyte infiltrating into tissues from the blood is in an inactive state (resting state), although the monocyte is differentiated into the macrophage.

Substrate

In the cell culture substrate for trait induction control of a macrophage of the embodiment, a substrate to be used has a pattern of unevenness on the base plate. In addition, the substrate is not particularly limited as long as the substrate is not deformed when culturing cells or by pretreatment such as sterilization treatment. The whole of the substrate may be composed of the same material, and the substrate may be composed of a pattern of unevenness made of different materials, and a base plate for supporting the pattern of unevenness.

Examples of the form of the substrate include a multi-well plate, a petri dish, and the like, on which any number of wells are disposed. Examples of the number of wells include 6, 12, 24, 96, 384, 1,536, or the like per plate.

Base Plate

In a case where the substrate may be composed of the pattern of unevenness made of different materials, and the base plate for supporting the pattern of unevenness, the material of the base plate is not particularly limited as long as the base plate is used for cell culture applications. More specific examples of the material of the base plate include glass, polyethylene terephthalate, polycarbonate, cycloolefin polymer, polydimethylsiloxane, polystyrene, and the like. By using these materials, autofluorescent materials can be reduced, and the cultured cells can be observed with a fluorescence microscope.

Uneven Pattern

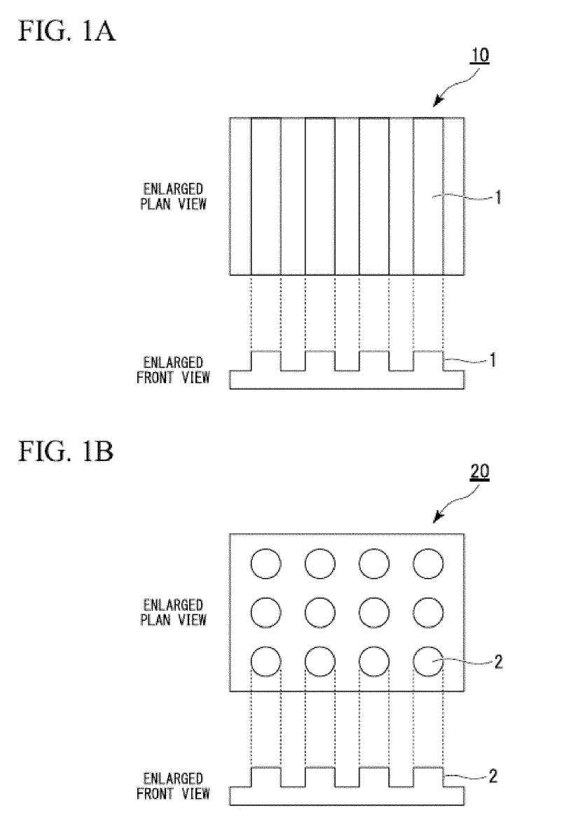

Examples of patterns of unevenness include a lattice shape, a radial shape, a polygon continuous shape on a flat surface (for example, a honeycomb structure or the like), a labyrinthine shape, a line shape, a dot shape, or the like. FIGS. 1A and 1B are an enlarged plan view and an enlarged front view of the cell culture base member for trait induction control of a macrophage according to the embodiment. FIG. 1A illustrates a case where the pattern of unevenness is a line shape, and FIG. 1B illustrates a case where the pattern of unevenness is a dot shape. In the enlarged front view of FIG. 1A, the shape of a projection portion is a rectangular parallelepiped line shape, but it is not limited thereto, and the shape of the projection portion may be a rectangular column shape (including a rectangular parallelepiped and a cube in a rectangular column), a truncated pyramidal shape, a semicircular column shape (including a semi-elliptical column in a semicircular column), a truncated conical shape (including an elliptical frustum and a biconical truncated cone in a truncated cone), or the like.

In addition, FIG. 1B illustrates a dot shape in which the transverse section of the projection portion is a circular shape and the vertical section of the projection portion is a rectangular shape (that is, the shape of the projection portion is circular column shape), but it is not limited thereto, and the transverse section or the vertical section of the projection portion may be a polygonal shape such as triangle and square, a circular shape (including a substantially circular shape, an elliptical shape, a substantially elliptical shape, a semicircular shape, and a fan shape in a circular shape), a trapezoidal shape, a wave shape, or the like. That is, examples of the shape of the projection portion include a rectangular column shape (including a rectangular parallelepiped and a cube in a rectangular column), a truncated pyramidal shape (including a truncated bipyramid in a truncated pyramid), a circular column shape (including an elliptic column, a semicircular column, a semi-elliptical column, and a sectoral column in a circular column), and a truncated conical shape (including an elliptical frustum and a biconical truncated cone in a truncated cone), or the like, but it is not limited thereto.

The width of the concave portion and the projection portion is preferably 50 nm or more and 1,000 nm or less, more preferably 100 nm or more and 1,000 nm or less, and further preferably 150 nm or more and 1,000 nm or less. When the width is within the above range, it is possible to stimulate the cell surface of the macrophage and to easily and efficiently induce the macrophage to a certain trait. In a case where the shape of the projection portion is a circular column shape or a truncated conical shape, the width represents the diameter of the upper surface of the projection portion.

The distance to the surface of the projection portion of the uneven pattern is preferably 10 nm to 100 μm. In a case where the distance to the surface of the projection portion is within the above range, autofluorescence of the substrate is easily suppressed. Therefore, when the substrate having the distance within the above range is used, it is easy to observe the cultured cells by the fluorescence microscope.

Method of Forming Uneven Pattern

A method of forming the uneven pattern is not particularly limited. Examples of the method of forming the uneven pattern include a photolithography method in which a photosensitive composition layer formed on a surface of a substrate for supporting an uneven pattern is selectively exposed, and thereafter a portion corresponding to a concave portion is removed from the exposed photosensitive composition layer with a developing solution, an imprinting method of curing an imprint material after pressing a pressing mold having a pattern of unevenness on a layer of the imprint material formed on a base plate surface, a method in which a mask for covering a portion corresponding to a projection portion is provided on a base plate surface, and thereafter a concave portion is formed on the base plate surface by a chemical treatment such as etching, a method of grinding a base plate surface by sand blasting or various machine tools, a method of attaching a material constituting a projection portion of a pattern having a predetermined shape to a base plate surface, and the like. For the photolithography method and the imprinting method, a photosensitive resin composition used for various purposes in the related art and a photosensitive spin-on-glass (SOG) material can be used without particular limitation.

Photosensitive Resin Composition

Examples of the photosensitive resin composition used for forming the uneven pattern include a photosensitive resin composition containing a resin component, a cationic polymerization initiator, and a solvent, and the like. The photosensitive resin composition may be any of a positive type and a negative type.

The resin component is not particularly limited as long as the resin component can be used for cell culture, for example. Among these, a polymer of a compound having an ethylenic unsaturated bond is preferable. Examples of the polymerizable functional group contained in the compound having an ethylenic unsaturated bond include a (meth)acryloyl group, a vinyl group, an allyl group, and the like. As the compound having the ethylenic unsaturated bond, for example, a monofunctional, a difunctional, or a trifunctional or higher polyfunctional, (meth)acrylate compound, (meth)acrylamide compound, vinyl compound, allyl compound, or the like can be used. These compounds having the ethylenic unsaturated bond can be used alone or in a combination of two or more.

Examples of the polyfunctional compound having the ethylenic unsaturated bond include trifunctional or higher acrylates such as trimethylolpropane tri(meth)acrylate, pentaerythritol tetra(meth)acrylate, ethylene oxide modified pentaerythritol tetra(meth)acrylate, propylene oxide modified pentaerythritol tetra(meth)acrylate, dipentaerythritol penta(meth)acrylate, dipentaerythritol hexa(meth)acrylate, and the like; a polyfunctional urethane (meth)acrylate obtained by reacting a polyisocyanate compound and a hydroxy group-containing (meth)acrylate monomer; and a condensate of polyhydric alcohol and N-methylol(meth)acrylamide, and the like. These polyfunctional compounds can be used alone or in a combination of two or more.

Examples of the difunctional compound having the ethylenic unsaturated bond include polyethylene glycol di(meth)acrylate, polypropylene glycol di(meth)acrylate, polyethylene polypropylene glycol di(meth)acrylate, ethylene glycol di(meth)acrylate, propylene glycol di(meth)acrylate, polyethylene poly trimethylolpropane di(meth)acrylate, 2-(meth)acryloyloxy-2-hydroxypropyl phthalate, 2-(meth)acryloyloxyethyl-2-hydroxyethyl phthalate, a compound obtained by reacting a glycidyl group-containing compound with α,β-unsaturated carboxylic acid, urethane monomers, γ-chloro-β-hydroxypropyl-β′-(meth)acryloyloxyethyl-o-phthalate, β-hydroxyethyl-β′-(meth)acryloyloxyethyl-o-phthalate, β-hydroxypropyl-β′-(meth)acryloyloxyethyl-o-phthalate, and the like.

Examples of the compound obtained by reacting the glycidyl group-containing compound with α,β-unsaturated carboxylic acid include triglycerol di(meth)acrylate, and the like. Examples of the urethane monomer include addition reaction products of a (meth)acrylic monomer having a hydroxyl group at the β position with isophorone diisocyanate, 2,6-toluene diisocyanate, 2,4-toluene diisocyanate, 1,6-hexamethylene diisocyanate, or the like, EO modified urethane di(meth)acrylate, EO,PO modified urethane di(meth)acrylate, and the like.

Examples of monofunctional compounds having the ethylenic unsaturated bond include (meth)acrylic acid esters, (meth)acrylamides, allyl compounds, vinyl ethers, vinyl esters, styrenes, and the like. These compounds can be used alone or in a combination of two or more.

Examples of the (meth)acrylate esters include methyl (meth)acrylate, ethyl (meth)acrylate, propyl (meth)acrylate, amyl (meth)acrylate, t-octyl(meth)acrylate, chloroethyl (meth)acrylate, 2,2-dimethylhydroxypropyl (meth)acrylate, 2-hydroxyethyl (meth)acrylate, trimethylolpropane mono(meth)acrylate, benzyl (meth)acrylate, furfuryl (meth)acrylate, phenyl (meth)acrylate, (meth)acrylate of EO adduct of phenol, (meth)acrylate of PO adduct of phenol, (meth)acrylate of EO/PO co-adduct of phenol, ethylene glycol mono(meth)acrylate, diethylene glycol mono(meth)acrylate, triethylene glycol mono(meth)acrylate, polyethylene glycol mono(meth)acrylate, 2-methoxyethyl (meth) acrylate, diethylene glycol monomethyl ether mono(meth)acrylate, triethylene glycol monomethyl ether mono(meth)acrylate, polyethylene glycol monoethyl ether mono(meth)acrylate, propylene glycol mono(meth)acrylate, dipropylene glycol mono(meth)acrylate, tripropylene glycol mono(meth)acrylate, polypropylene glycol mono(meth)acrylate, propylene glycol monomethyl ether mono(meth)acrylate, dipropylene glycol monomethyl ether mono(meth)acrylate, tripropylene glycol monomethyl ether mono(meth)acrylate, polypropylene glycol monomethyl ether mono(meth)acrylate, mono(meth)acrylate of EO/PO copolymer, monomethyl ether mono(meth)acrylate of EO/PO copolymer, and the like.

Examples of the (meth)acrylamides include (meth)acrylamide, N-alkyl (meth)acrylamide, N-allyl (meth)acrylamide, N,N-dialkyl (meth)acrylamide, N,N-allyl (meth)acrylamide, N-methyl-N-phenyl (meth)acrylamide, N-hydroxyethyl-N-methyl (meth)acrylamide, and the like.

Examples of the vinyl ethers include alkyl vinyl ethers such as hexyl vinyl ether, octyl vinyl ether, decyl vinyl ether, ethylhexyl vinyl ether, methoxyethyl vinyl ether, ethoxyethyl vinyl ether, chloroethyl vinyl ether, 1-methyl-2,2-dimethylpropyl vinyl ether, 2-ethyl butyl vinyl ether, hydroxyethyl vinyl ether, diethylene glycol vinyl ether, dimethylamino ethyl vinyl ether, diethyl aminoethyl vinyl ether, butyl aminoethyl vinyl ether, benzyl vinyl ether, tetrahydrofurfuryl vinyl ether, and the like; vinyl allyl ethers such as vinyl phenyl ether, vinyl tolyl ether, vinyl chlorophenyl ether, vinyl-2,4-dichlorphenyl ether, vinyl naphthyl ether, vinyl anthranyl ether, and the like.

Examples of the vinyl esters include vinyl butyrate, vinyl isobutyrate, vinyl trimethyl acetate, vinyl diethyl acetate, vinyl valate, vinyl caproate, vinyl chloroacetate, vinyl dichloroacetate, vinyl methoxy acetate, vinyl butoxy acetate, vinyl phenylacetate, vinyl acetoacetate, vinyl lactate, vinyl-β-phenyl butyrate, vinyl benzoate, vinyl salicylate, vinyl chlorobenzoate, vinyl tetrachlorobenzoate, vinyl naphthoate, and the like.

Examples of the styrenes include styrene; alkyl styrene such as methyl styrene, dimethyl styrene, trimethylstyrene, ethylstyrene, diethylstyrene, isopropylstyrene, butylstyrene, hexylstyrene, cyclohexylstyrene, decylstyrene, benzylstyrene, chloromethylstyrene, trifluoromethylstyrene, ethoxymethylstyrene, acetoxymethylstyrene, and the like; alkoxystyrene such as methoxystyrene, 4-methoxy-3-methylstyrene, dimethoxystyrene, and the like; halostyrene such as chlorostyrene, dichlorostyrene, trichlorostyrene, tetrachlorostyrene, pentachlorostyrene, bromostyrene, dibromostyrene, iodostyrene, fluorostyrene, trifluorostyrene, 2-bromo-4-trifluoromethylstyrene, 4-fluoro-3-trifluoromethylstyrene, and the like.

The cationic polymerization initiator is one that generates a cation upon irradiation with radiation such as ultraviolet ray, far ultraviolet ray, an excimer laser such as KrF, ArF or the like, X-ray and electron beam, and the like, and the cation thereof is a compound that can be a polymerization initiator.

As the cationic polymerization initiator, for example, an onium salt type cationic polymerization initiator such as an iodonium salt or a sulfonium salt can be used. The anion to be a counter ion of the onium ion constituting the onium salt type cationic polymerization initiator is preferably a fluorinated alkyl fluorophosphate anion, a hexafluorophosphate anion, or a hexafluoroantimonate acid anion (SbF₆—).

The solvent contained in the photosensitive resin composition is not particularly limited as long as the solvent can prepare a uniform photosensitive resin composition and does not hinder the effect of exposure. The boiling point of the solvent is preferably 50° C. to 200° C.

Specific examples of the solvent include aliphatic hydrocarbons such as hexane, heptane, octane, decane, and cyclohexane; alcohols such as methanol, ethanol, 1-propanol, 2-propanol, and 1-butanol; acetone, methyl ethyl ketone, methyl isobutyl ketone, 2-heptanone, ethyl lactate, propylene glycol monomethyl ether acetate, propylene glycol monomethyl ether, ethyl acetate, butyl acetate, ethylene glycol dimethyl ether, propylene glycol dimethyl ether, methyl cellosolve, ethyl cellosolve, dibutyl ether, methyl-3-methoxypropionate, propylene glycol mono propyl ether, butyl cellosolve, diethylene glycol diethyl ether, hexylene glycol, cyclohexanone, propylene glycol monoethyl ether, ethyl pyruvate, ethyl cellosolve acetate, and the like. These may be used alone or in a combination of two or more.

In addition to the resin component, the cationic polymerization initiator, and the solvent, the photosensitive resin composition may contain various additives used in the photosensitive resin composition in the related art. Examples of such additives include additional resins, sensitizers, plasticizers, stabilizers, colorants, coupling agents, leveling agents, and the like.

The photosensitive resin composition can be prepared by mixing (dispersing and kneading) each of the above components with a stirrer such as a triple roll mill, a ball mill, a sand mill, or the like and filtering with a filter such as a 5 μm membrane filter if required.

Method of Preparing Substrate

The method of preparing the substrate is not particularly limited as long as it is a method capable of forming the substrate having a desired pattern by exposing and curing the above-described photosensitive resin composition. As a method of preparing a cell culture substrate, for example, a method including a coating process of coating a photosensitive resin composition onto a base plate to form a coating film and an exposure process of exposing the coating film on the base plate to cure the coating film can be mentioned. The method of preparing the substrate may include a detachment process of detaching the exposed coating film from the base plate after curing the coating film on the base plate by exposure if required.

In the coating process, the base plate on which the photosensitive resin composition is coated is not particularly limited as long as the base plate does not cause deformation or deterioration in the process of preparing the substrate. As the material of the base plate, the same base plate as the above “Base plate” can be mentioned.

As a method of forming a pattern of unevenness, the same method as the above “Method of Forming Uneven Pattern” can be mentioned.

The method of forming the coating film on the base plate is not particularly limited, and examples thereof include a method in which a predetermined amount of the photosensitive resin composition is dropped onto the base plate, a method of using a contact transfer type coating applicator such as a roll coater, a reverse coater, a bar coater, or the like, and a method of using a non-contact type coating applicator such as a spinner (rotary coating applicator), a curtain flow coater, or the like.

After the coating film is formed, the base plate provided with the coating film may be placed under a reduced pressure condition to degas the coating film.

In the exposure process, the method of exposing the coating film is not particularly limited as long as the coating film can be satisfactorily cured. For the exposure, for example, a light source emitting ultraviolet rays such as a high-pressure mercury vapor lamp, an ultrahigh pressure mercury vapor lamp, a xenon lamp, a carbon arc lamp or the like may be used. The exposure amount at the time of exposing the coating film is appropriately determined in consideration of the composition of the photosensitive resin composition, the film thickness of the coating film, and the like. Typically, the exposure amount when the coating film is exposed is preferably 10 to 100,000 mJ/cm², and more preferably 100 to 50,000 mJ/cm².

The method of exposing the coating film is not particularly limited, but the coating film may be first exposed to the atmosphere to partially cure the coating film. In this manner, in the exposure process, it is possible to prevent the photosensitive resin composition from protruding from the base plate, and thereafter to expose the coating film in water. If the coating film is exposed in water without exposure to the atmosphere, the coating film may dissolve in water in some cases. When the coating film is exposed to the atmosphere and thereafter the coating film is exposed in water, radical polymerization inhibition due to oxygen can be reduced and a good cured film can be obtained.

In addition, the exposure process may include exposure of the coating film in a vacuum. When the coating film is exposed to the vacuum, the coating film of the photosensitive resin composition can be cured in a state of being in close contact with the base plate, and a substrate having a desired pattern is easily formed. In addition, in a case where the coating film is exposed to the vacuum, exposure may be performed while applying pressure to the coating film from the upper surface of the base plate. In this case, the coating film of the photosensitive resin composition can be cured in a state of being in close contact with the base plate. When the exposure process includes exposure to the vacuum or exposure to the vacuum while applying pressure, specifically, in a case where a substrate is formed by using a mold corresponding to the pattern of unevenness provided in the substrate, it is possible to accurately transfer the uneven pattern of the mold to the substrate. By exposing the coating film under such conditions, shrinkage upon curing of the photosensitive resin composition is suppressed, so that the uneven pattern of the mold can be accurately transferred to the substrate.

As a method of exposing the coating film to the vacuum, for example, a method in which the surface of the coating film is coated with a film such as a PET film, and thereafter the coating film is exposed at least in a state where a space between the film and the coating film is vacuumed can be mentioned. In a case of exposing while applying pressure to the coating film, as a method of applying pressure to the coating film, for example, a method such as negative pressure exposure can be mentioned.

The coating film that is exposed and cured by the method as described above is used as the substrate after detaching from the mold if required.

In addition, the exposed and cured coating film may be subjected to a plasma treatment. By subjecting the cured coating film to the plasma treatment, it is possible to form the substrate to which the cell is likely to adhere. Plasma used for the plasma treatment is not particularly limited, but examples thereof include O₂ plasma, N₂ plasma, CF₄ plasma, and the like. The timing of the plasma treatment is not particularly limited, and the plasma treatment may be performed at any timing before or after detaching the cured coating film from the base plate.

Furthermore, the substrate detached from the mold may be rinsed with a rinsing liquid. When the substrate is rinsed with the rinsing liquid, a compound which can cause cytotoxicity such as an unreacted photopolymerizable monomer or photopolymerization initiator can be removed from the surface of the substrate. Examples of the rinsing liquid include organic solvents such as propylene glycol-1-methyl ether acetate (PGMEA), isopropyl alcohol (IPA), and acetone, water, and the like.

Application

In addition, a medical material with improved biocompatibility can be obtained by processing the cell culture substrate for trait induction control of a macrophage of the embodiment on the surface of the medical material. Furthermore, by using the medical material, differentiation of the macrophage can be controlled and tissues can be effectively regenerated.

Examples of medical materials include a medical molded body for scaffold used for tissue regeneration or transplantation tissue formation such as heart, blood vessel, cartilage, skin, kidney, liver, myocardium, muscle, tendon, and the like; a medical molded body for implanting in a living body such as aneurysm coil, embolic material, artificial mucosa, artificial esophagus, artificial trachea, artificial blood vessel, artificial valve, artificial chest wall, artificial pericardium, artificial heart muscle, artificial diaphragm, artificial peritoneum, artificial ligament, artificial tendon, artificial skin, artificial joint, artificial cartilage, and the like; surgical suture, surgical prosthetic material, surgical reinforcing material, wound protecting material, bone fracture bonding material, catheter, syringe, infusion bag or blood bag, blood filter, material for extracorporeal circulation, and the like, but the medical material is not limited thereto.

Method of Controlling Trait of Macrophage

In one embodiment, the present invention provides a method of controlling trait of a macrophage in which the macrophage is cultured on the cell culture substrate for trait induction control of a macrophage as described above.

According to the method of controlling trait of a macrophage of the embodiment, the macrophage can be easily and efficiently induced to a certain trait. In addition, the macrophage can be induced into the inflammatory type or anti-inflammatory type macrophage, and disease models and the like can be easily constructed.

Culturing Process

In the trait control method of the embodiment, the macrophage is cultured using the above-described cell culture substrate for trait induction control of a macrophage.

As the macrophage to be used, the same macrophage as the above-described “Macrophage” can be mentioned. In addition, the macrophage may be differentiated from the above-described monocyte.

The culture medium to be used may be a basic culture medium containing components (inorganic salts, carbohydrates, hormones, essential amino acids, non-essential amino acids, and vitamins) and the like required for the cell's viable growth. Examples of the culture medium include Dulbecco's Modified Eagle's Medium (DMEM), Minimum Essential Medium (MEM), Basal Medium Eagle (BME), Dulbecco's Modified Eagle's Medium: Nutrient Mixture F-12 (DMEM/F-12), Glasgow Minimum Essential Medium (Glasgow MEM), Gibco® RPMI 1640 culture medium (manufactured by Life Technologies), HL-1 known composition, serum-free culture medium (manufactured by Lonza Inc.), and the like. In the culturing process, the culture medium may be suitably replaced with a new one according to the growth rate of the cells.

In addition, a compound inducing the differentiation or trait of the macrophage may be added to the culture medium to be used. By adding the compound, the rate of differentiation or trait change can be further accelerated, and differentiation or trait can be controlled in a certain direction. Examples of compounds that trait-induce the macrophage into the M1 macrophage include Th1 cytokines such as interferon (IFN)-γ, tumor necrosis factor (TNF)-α, lipopolysaccharide (LPS) and the like, and two or more of these compounds may be used in combination. In addition, examples of compounds that trait-induce the macrophage into the M2 macrophage include Th2 cytokines such as interleukin (IL)-4 and IL-13, and two or more of these compounds may be used in combination. In addition, the compounds trait-inducing into the M1 macrophage and the compounds trait-inducing into the M2 macrophage may be used in combination.

The concentration of the compounds that induce the macrophage differentiation is not particularly limited, and may be 1 nM or more and 1 μM or less, and may be 5 nM or more and 100 nM or less. Within the above range, it is possible to more efficiently induce the trait from the macrophage into the M1 or M2 macrophage.

Culture conditions are not particularly limited as long as it is a method suitable for culturing the macrophage, for example, the density of seeding the macrophage in the culture medium is preferably 1×10⁰ to 1×10⁷ cells/mL, and more preferably 1×10² to 1×10⁶ cells/mL. The culture temperature is preferably 25° C. or more and 40° C. or less, more preferably 30° C. or more and 39° C. or less, and further preferably 35° C. or more and 39° C. or less. The culturing time can be appropriately set depending on the growth state of the macrophage, and it is preferably 1 hour or more and 100 hours or less. By using the cell culture substrate for trait induction control of a macrophage as described above, the trait induction of the macrophage is promoted, and the macrophage can be differentiated in a shorter time than the method in the related art. The culture environment is preferably cultured under CO₂ conditions through approximately 5% carbon dioxide.

Foam Cell

In the trait control method of the embodiment, the macrophage can be further trait-induced into a foam cell.

In the present specification, “foam cell” refers to a cell positive for CD36 serving as a receptor recognizing and treating oxidized LDL cholesterol, and formed after swallowing the oxidized LDL cholesterol. Since the accumulated oxidized LDL cholesterol appears to be foam particles in the cell, it is called a foam cell. It is known that the foam cell releases cytokines such as platelet growth factors and migrates and proliferates equilibrium myocytes present in the vascular media. Normally, the foam cell dies due to oxidative stress, but in a case where the foam cell is not rapidly removed due to deficiency of adiponectin or the like, the contents of the cell may leak out and cause inflammation. This inflammatory response brings a vicious circle of generating a new macrophage and further increasing the death of the foam cell, and necrosis centers are eventually formed by the accumulation of the foam cell carcasses. In the necrotic center, fibroblasts gather to repair the inflammation caused in blood vessels. It is due to the action of the fibroblast that wounds rise when the injury occurs and the scab is formed. Due to the action of fibroblasts, the necrosis center is consolidated with collagen, and necrosis center which is called atheroma plaque and nodules made of collagen wrapped therearound are formed. Therefore, the foam cell is often observed in arteriosclerosis (mushy shape atheroma).

Therefore, in the method of controlling trait of the embodiment, the disease model such as arteriosclerosis or the like can be easily constructed by inducing trait to the foam cell.

### EXAMPLES

Hereinafter, the present invention will be described with reference to Examples, but the present invention is not limited to the following Examples.

### Preparation Example 1

Preparation of Substrate by Directed Self-Assembly (DSA)

(1) 0.2 mL of a propylene glycol monomethyl ether acetate solution containing 2 wt % of a block copolymer (number average molecular weight 18,000-b-18,000) of polystyrene and polymethyl methacrylate was dropped on a smooth surface of one sheet of a 0.8 cm×0.8 cm glass base plate (manufactured by Hiraoka Specialty Glass Co., Ltd.) to form a coating film on the base plate. Subsequently, the glass base plate on which the coating film was formed was annealed at 240° C. for 60 seconds. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.), and the polymethyl methacrylate portion was selectively dry-etched to obtain a substrate (LS1). In addition, except for using a block copolymer (number average molecular weight 49,000-b-21,000) of polystyrene and polymethyl methacrylate, the same treatment was performed on the smooth surface of one sheet of a 0.8 cm×0.8 cm glass base plate (manufactured by Hiraoka Specialty Glass Co., Ltd.) to obtain a substrate (P1).

(2) 0.2 mL of a 2 wt % propylene glycol monomethyl ether acetate solution of polystyrene (number average molecular weight 18,000) was dropped on a smooth surface of one sheet of a 0.8 cm×0.8 cm glass base plate (manufactured by Hiraoka Specialty Glass Co., Ltd.) and one sheet of a 0.8 cm×0.8 cm polyethylene terephthalate (PET) base plate (manufactured by Mitsubishi Chemical Corporation) to form a coating film on the respective base plates. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.) to obtain substrates (Smooth 1 and Smooth 2).

TABLE 1

| | Smooth 1 | LS 1 | P 1 | Smooth 2 | Smooth 3 | P 2 | P 3 | P 4 | P 5 | LS 2 | LS 3 | LS 4 | LS 5 | LS 6 | LS 7 | LS 8 |
|---|---|---|---|---|---|---|---|---|---|---|---|---|---|---|---|---|
| Type of | Glass | | | PET | | | | | | | | | | | | |
| base plate | | | | | | | | | | | | | | | | |
| Photosensitive | Polystyrene | | | | Acrylic resin | | | | | | | | | | | |
| resin composition | | | | | (Radical negative resist) | | | | | | | | | | | |
| Pattern | — | Line | Pillar | — | — | Pillar | | | | Line and Space | | | | | | |
| | | and | | | | | | | | | | | | | | |
| | | Space | | | | | | | | | | | | | | |
| Width (nm) | 0 | 14 | 20 | 0 | 0 | 100 | 200 | 300 | 500 | 75 | 150 | 200 | 250 | 300 | 500 | 1,000 |

In Table 1, “Smooth” represents a flat substrate used as a control, on which an uneven pattern is not formed. In addition, “Width” represents the width of the projection portion.

### Preparation Example 2

Preparation of Substrate by Transfer from Argon Fluoride (ArF) Pattern

A radical polymerization negative resist containing a photosensitive resin composition containing an acrylic resin as a main component was used as a photoresist composition. 1 ml of the radical polymerization negative resist was dropped on a 0.8 cm×0.8 cm silicon wafer having a pattern of unevenness (Smooth 3, P2 to P5, and LS2 to LS8) illustrated in Table 1 formed using ArF exposure machine Nikon 5308, the coating film was degassed by placing the coating film under a reduced pressure condition of 100 Pa for 30 minutes, and a radical polymerization negative resist was embedded in the uneven pattern. Subsequently, twelve silicon base plates each having the coating film were exposed in an atmosphere with an exposure amount of 999 J/m² using an ultraviolet irradiation device (HMW-532D, manufactured by ORC Co., Ltd). Subsequently, the film cured by exposure as described above was covered with a base plate prepared by coating a radical polymerization negative resist having a film thickness of 1 μm onto a PET base plate (manufactured by Mitsubishi Chemical Corporation) so that the radical polymerization negative resist having a film thickness of 1 μm was in contact with the film cured, and exposure with an exposure amount of 999 J/m² was repeated five times using an ultraviolet irradiation device (HMW-532D, manufactured by ORC Co., Ltd) in a vacuum, to cure the coating film and the radical polymerization negative resist having a film thickness of 1 μm. After detaching the mold from the cured coating film, the cured coating film was immersed in propylene glycol-1-methyl ether acetate (PGMEA) for 10 minutes and rinsed, and thereafter the nitrogen gas was blown onto the cured coating film to be dried. Subsequently, O₂ plasma treatment was performed on the dried cured coating film under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Ohka Kogyo Co., Ltd.) to obtain a substrate (Smooth 3, P2 to P5 and LS2 to LS8).

### Example 1

Trait Control of Macrophage

(1) Culture of Macrophage

A cell culture test was performed at a culture temperature of 37° C. and 5% CO₂ environment using the substrate obtained in Preparation Examples 1 and 2. RAW264 cell derived from a mouse macrophage (using RIKEN Bank cells, RIKEN Bank RCB 0535) was used as the cell to be cultured. As a culture medium, RPMI 1640 culture medium containing 10% bovine serum (Fetal Bovine Serum; FBS) was used. The substrate obtained in Preparation Examples 1 and 2 was placed in a well of a dish with a well, and thereafter 2×10⁴ cells per one substrate were seeded on the surface of the substrate. Thereafter, the culture medium was injected into the well with a disposable pipette and cultured for 1 day.

As a result of observing a form of the cells after 1 day culture, it was confirmed that RAW264 cells were transformed into an elongated form in a case where a substrate of LS6 (line and space pattern, width of 300 nm) was used. Generally, it is known that the resting macrophage has a rounded form and changes into an elongated form when differentiated into an inflammatory type. Therefore, it was suggested that the macrophage can be differentiated without using trait inducing compounds by culturing using the substrate of LS6.

(2) Differentiation of Macrophage

Subsequently, trait inducing compounds were added to a portion of cultured RAW264 cells. Lipopolysaccharide (LPS) and interferon gamma (IFNγ) derived from Escherichia coli were used as compounds that trait-induce into the M1 macrophage serving as the inflammatory type. In addition, interleukin 4 (IL-4) was used as a compound that trait-induces into the M2 macrophage serving as the anti-inflammatory type. The compounds were added so that the concentration of each compound in the culture medium was 10 ng/mL, and cultured for 1 day.

(3) Quantifying of Amount of Nitric Oxide (NO) Production

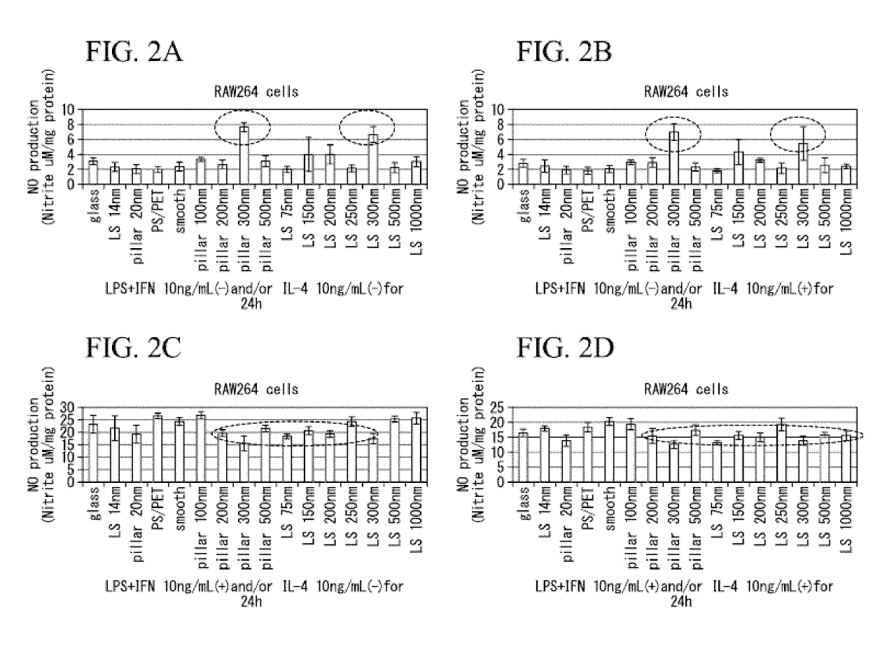

An equal amount of 10% grease reagent was added to the culture solution of RAW264 cells cultured in (2) and allowed to react at room temperature for 10 minutes. In the inflammatory macrophage, nitric oxide (NO) is synthesized from arginine, and the synthesized NO is oxidized to NO₂. Furthermore, NO₂ reacts with water to become nitrous acid or nitric acid. In the method using the grease reagent, it is possible to indirectly evaluate the amount of NO production by quantifying the generated nitrous acid. Subsequently, absorbance at 540 nm was measured using a microplate reader. A calibration curve was prepared in advance with a nitrous acid standard solution, and the concentration of nitrous acid in the culture supernatant was calculated. The results are illustrated in FIGS. 2A to 2D.

From FIGS. 2A to 2D, in a case where LPS and IFNγ were added, the amount of NO production decreased in a portion of the substrates. In a case of using the substrates of P4 (pillar pattern, width of 300 nm) and LS6 (line and space pattern, width of 300 nm), it was confirmed that the amount of NO production increased in the absence of LPS and IFNγ.

From the above, it was confirmed that the M1 macrophage serving as the inflammatory type was trait-induced by using the substrates of P4 (pillar pattern, width of 300 nm) and LS6 (line and space pattern, width of 300 nm).

(4) Evaluation of Arginase Activity

(4-1) Enzyme Substrate Reaction Process

A portion of the RAW264 cells cultured in (2) was disrupted to prepare a cell-disrupted liquid as a sample containing arginase. Subsequently, 20 μL of the cell-disrupted liquid was dispensed to the microplate.

Subsequently, 20 μL of Mn solution (5 mM MnCl₂·25 nM TrisHCl (pH 7.5)) was dispensed to activate the enzyme. Subsequently, 40 μL of arginine buffer (0.5 M arginine (pH 9.7)) was dispensed to initiate enzyme substrate reaction. In addition, wells containing only the cell-disrupted liquid were prepared as a blank without dispensing 2 times concentrated substrate buffer. The mixture was incubated at 37° C. for 2 hours, and the enzyme substrate reaction was performed.

(4-2) Enzyme Inactivation and Urea Detection Reaction Process

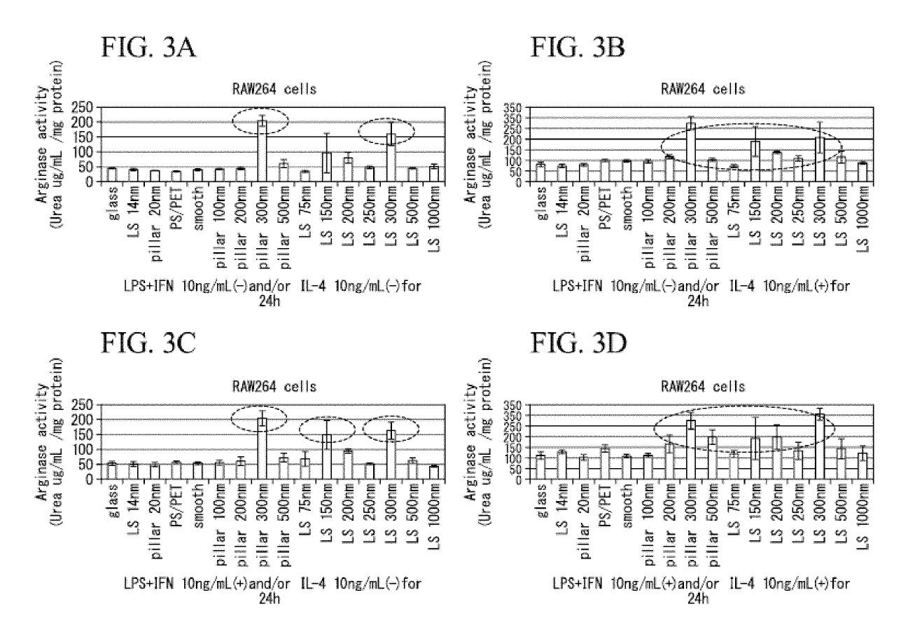

Subsequently, each 180 μL of a urea detection reagent (iso nitroso propiophenone 9 wt %/phosphoric acid:concentrated sulfuric acid:water=1:3:7 (volume ratio)) was added to the microplate, the enzyme reaction was stopped, and the urea detection reaction was started. Subsequently, the mixture was incubated at 95° C. for 1 hour and 45 minutes. Subsequently, the absorbance at 540 nm of a sample containing arginase was measured using a microplate reader (Spectra Max i3 manufactured by Molecular Devices, LLC) in which a calibration curve was drawn with urea solutions of 0, 5, 10, 20, 40, 80 and 160 μg/mL in advance. The results are illustrated in FIGS. 3A to 3D.

From FIGS. 3A to 3D, in a case of using the substrates of P4 (pillar pattern, width of 300 nm), LS3 (line and space pattern, width of 150 nm) and LS6 (line and space pattern, width of 300 nm), it was confirmed that arginase activity increased in the absence of IL-4.

From the above, it was confirmed that the M2 macrophage serving as the anti-inflammatory type was trait-induced by using the substrates of P4 (pillar pattern, width of 300 nm), LS3 (line and space pattern, width of 150 nm) and LS6 (line and space pattern, width of 300 nm).

### Example 2

Trait Induction of Macrophage to Foam Cell

(1) Culture of Macrophage

A cell culture test was performed at a culture temperature of 37° C. and 5% CO₂ environment using the substrate obtained in Preparation Examples 1 and 2. RAW264 cell derived from a mouse macrophage (using RIKEN Bank cells, RIKEN Bank RCB 0535) was used as the cell to be cultured. As a culture medium, RPMI 1640 (22400-089 manufactured by GIBCO) containing 10% FBS was used. The substrate obtained in Preparation Examples 1 and 2 was placed in a well of a dish with a well, and thereafter 2×10⁴ cells per one substrate were seeded on the surface of the substrate. Thereafter, the culture medium was injected into the well with a disposable pipette and cultured for 24 hours or 48 hours.

(2) Fixation of Macrophage

The cells were fixed by using a 4% paraformaldehyd (PFA) solution (solvent: PBS) and placing the solution at room temperature for 10 minutes. Subsequently, the 4% PFA solution was removed and washed with PBS.

(3) Cell Dyeing

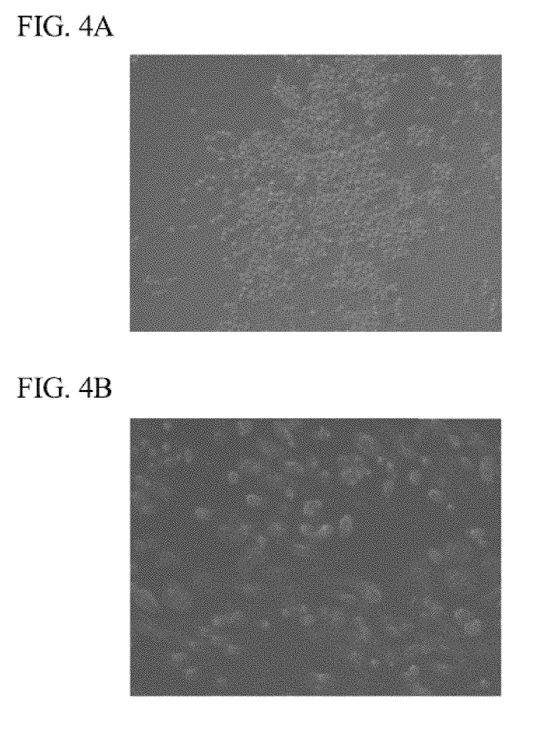

Subsequently, the solution was replaced by using 60% isopropanol (40% is purified water) and placing the solution at room temperature for 1 minute. Subsequently, 60% isopropanol was removed and the solution was dyed by using 60% Oil Red O (O-0625 manufactured by SIGMA, the solvent is isopropanol:purified water=6:4) and placing the solution at room temperature for 15 minutes. The Oil Red O dyes oil droplets in red. Subsequently, the 60% Oil Red O was removed, and after washing with purified water, cells were observed using an optical microscope (routine inverted microscope manufactured by Carl Zeiss microscope Co., Ltd., Germany). The results are illustrated in FIGS. 4A and 4B. FIG. 4A illustrates the result of culturing using the substrate “Smooth 3”. FIG. 4B illustrates the result of culturing using the substrate of LS6 (line and space pattern, width of 300 nm). Both FIGS. 4A and 4B are images magnified 200 times.

From FIGS. 4A and 4B, in a case of using the substrate of Smooth 3, cells dyed in red were not observed, but in a case of using the substrate of LS6, cells dyed in red were observed.

Therefore, it was confirmed that it was trait-induced to the foam cell by using the substrate of LS6 (line and space pattern, width of 300 nm).

According to the cell culture substrate for trait induction control of the present invention, the macrophage can be easily and efficiently induced to a certain trait. In addition, it can be applied to the improvement of biocompatibility by surface treating of medical materials. According to the method of controlling trait of a macrophage of the present invention, the macrophage can be induced into the inflammatory type or anti-inflammatory type macrophage, and disease models and the like can be easily constructed.

### EXPLANATION OF REFERENCES

1 . . . linear shape convex portion, 2 . . . dot shape convex portion, 10, 20 . . . base material

What is claimed is:

1. A cell culture substrate for trait induction control of a macrophage, which has a pattern of unevenness on a surface to which a cell adheres, the width of the unevenness being 50 nm or more and less than 1,000 nm.

2. The cell culture substrate for trait induction control of a macrophage according to claim 1,
wherein the pattern of unevenness is a lattice shape, a radial shape, a polygon continuous shape on a flat surface, a labyrinthine shape, a line shape, or a dot shape.

3. A method of controlling trait of a macrophage, comprising:
culturing a macrophage on the cell culture substrate for trait induction control of a macrophage according to claim 1.

4. The method of controlling trait of a macrophage according to claim 3,
wherein a culturing time of the macrophage is 1 hour or more and 100 hours or less.

5. The method of controlling trait of a macrophage according to claim 3,
wherein the macrophage is trait-induced into foam cell.
